(19) United States
(12) Patent Application Publication
(10) Pub. No.: US 2025/0331844 A1
(43) Pub. Date: Oct. 30, 2025

(54) APPARATUS AND ITS METHOD OF USE FOR MANIPULATING SUTURE THREAD DURING MINIMALLY INVASIVE SURGERY

(71) Applicants: Jaeah Kim, New York, NY (US); Jaejeong Kim, New York, NY (US); Jaemin Kim, New York, NY (US)

(72) Inventors: Jaeah Kim, New York, NY (US); Jaejeong Kim, New York, NY (US); Jaemin Kim, New York, NY (US)

(21) Appl. No.: 18/647,424

(22) Filed: Apr. 26, 2024

Related U.S. Application Data
(60) Provisional application No. 63/498,822, filed on Apr. 28, 2023.

(51) Int. Cl. A61B 17/04; A61B 17/00; A61B 17/29

(52) U.S. Cl. CPC A61B 17/0469; A61B 17/29; A61B 2017/00017; A61B 2017/00398; A61B 2017/00411; A61B 2017/00734; A61B 2017/2903; A61B 2017/2919; A61B 2017/2929; A61B 2017/2931; A61B 2017/294

(57) ABSTRACT

Disclosed herein are embodiments of an apparatus and its method of use for manipulating suture thread handling. An example apparatus can include a motor, a housing with the rotary motor enclosed therein, and a shaft. At the end of the shaft are a pair of jaws and a pair of rollers. When the user closes the jaws over suture thread, the user can press a trigger to engage the motor, thereby turning the pair of rollers and pulling and/or pushing the suture thread as desired.

### CROSS-REFERENCE

This application claims priority to U.S. provisional patent application No. 63/498,822, filed Apr. 28, 2023, the contents of which are incorporated herein in their entirety.

### BACKGROUND

### 1. Technical Field

The present disclosure relates to a suture thread manipulating apparatus, and more specifically to its method of use for manipulating suture thread during minimally invasive surgery.

### 2. Introduction

Suturing in a laparoscopic environment can be tedious and time-consuming due to factors such as altered depth perception, counterintuitive movements, and a limited working field. This can result in a longer duration of operation, which is associated with negative outcomes, including postoperative nausea and vomiting, thromboemboli, postoperative infection, postoperative core hypothermia, postoperative cardiopulmonary complications, and mortality.

### SUMMARY

Additional features and advantages of the disclosure will be set forth in the description that follows, and in part will be understood from the description, or can be learned by practice of the herein disclosed principles. The features and advantages of the disclosure can be realized and obtained by means of the apparatus and combinations particularly pointed out in the appended claims. These and other features of the disclosure will become more fully apparent from the following description and appended claims, or can be learned by the practice of the principles set forth herein.

An apparatus configured as disclosed herein can include: a rotary motor; a housing with the rotary motor enclosed therein; a pair of rollers; a shaft having a first end and a second end, the shaft coupled at the first end to the pair of rollers and at the second end to the housing; a pair of jaws, each jaw in the pair of jaws respectfully coupled to a roller in the pair of rollers, such that when pressed together each jaw in the pair of jaws engages with the other jaw, and each roller in the pair of rollers is pressed against the other roller; a trigger assembly sitting on the shaft which, when engaged, causes the rotary motor to move the pair of jaws and the pair of rollers towards one another, and a handle assembly coupled to the shaft, wherein when the handle assembly is squeezed and the trigger assembly is pulled: the rotary motor is engaged and rotates the shaft, causing at least one roller in the pair of rollers to manipulate a suture thread in a direction.

An apparatus configured as disclosed herein can include: a pair of rollers; a shaft having a first end and a second end, the shaft coupled at the first end to the pair of rollers and at the second end to a handle assembly; a pair of jaws, each jaw in the pair of jaws respectfully coupled to a roller in the pair of rollers, such that when pressed together each jaw in the pair of jaws engages with the other jaw, and each roller in the pair of rollers is pressed against the other roller; a trigger assembly sitting on the shaft which, when engaged, causes the a motor to move the pair of jaws and the pair of rollers towards one another, and the handle assembly coupled to the shaft, wherein when the handle assembly is squeezed and the trigger assembly is pulled: the motor is engaged and rotates the shaft, causing at least one roller in the pair of rollers to manipulate a suture thread in a direction.

### BRIEF DESCRIPTION OF THE DRAWINGS

FIG. 1 illustrates a first example apparatus for manipulating suture thread;

FIG. 2 illustrates an expanded view of the apparatus illustrated in FIG. 1;

FIG. 3 illustrate a second example apparatus for manipulating suture thread;

FIG. 4. illustrates an example of jaws and rollers as used in a third example apparatus;

FIG. 5. illustrates a fourth example apparatus for manipulating suture thread;

FIG. 6. illustrates an example of jaws and rollers as used in a fifth example apparatus;

FIG. 7. illustrates a sixth example apparatus for manipulating suture thread;

FIG. 8. illustrates a seventh example apparatus for manipulating suture thread;

FIG. 9 illustrates a first exemplary method embodiment;

FIG. 10 illustrates a second exemplary method embodiment;

FIG. 11 illustrates an eighth example apparatus for manipulating suture thread;

FIG. 12 illustrates a ninth example apparatus for manipulating suture thread;

FIG. 13A illustrates a tenth example apparatus for manipulating suture thread; and

FIG. 13B illustrates the tenth example apparatus for manipulating suture thread.

### DETAILED DESCRIPTION

The following description of the preferred embodiments of the invention is not intended to limit the invention to these preferred embodiments, but rather to enable any person skilled in the art to make and use this invention. Disclosed herein is an apparatus and its method of use for manipulating suture thread. The apparatus and its method can be, for example, used in surgery. Non-limiting examples of surgery where the disclosed apparatus can be deployed can include minimally invasive surgery including laparoscopic, endoscopic, and robot surgery, to name a few.

Suturing consists of three main steps: taking a bite of the tissue using the needle, pulling the suture thread through the tissue, and making a knot. Existing solutions in the field focus on helping with the first and last step: pushing the needle through tissue or making a knot. However, there is an unmet need regarding the intermediate process of pulling the suture thread through tissue in a constrained working environment. Thus, there is a need for a new and useful system and method for manipulating suture thread during minimally invasive surgery.

The apparatus may include one or more rollers, a rotary motor attached to a shaft, a flexible shaft coupler, a bracing element, opposing jaw members, a trigger, and/or a handle. The handles may be attached to an elongated body that controls the movement of the opposing jaw members. Suture thread can be grasped with the rollers either manually or through the activation of a detachable motor.

Use of the apparatus may include grasping the suture thread with the rollers, engaging the rollers to reel suture thread through the tissue, the rollers disengaging automatically once a desired tension is reached in order to provide haptic feedback to the user, and repeating for each bite of the suture. Consider the following example. After a needle has been pushed through the tissue, the user needs to grasp the suture thread using the apparatus. The handles, resembling the handles/rings of scissors, can be used to open the rollers in open ready position to grasp the suture thread. Once in position, the user closes the handles, causing the suture thread to be grasped by the rollers. As the user presses a trigger or button, a motor can begin spinning the rollers, such that the suture thread will loop around the rollers. The grooves structure on the roller jaw further helps secure the grasping of the suture thread, pulling the suture thread taut as needed. The user can then continue putting the suture needle through tissue and pulling the thread taut, as needed until a knot needs to be tied. In some configurations, rather than the user opening and closing the rollers manually using scissor-like handles, the apparatus can have an automatic and/or motorized mechanism, allowing the user to press a button to cause the rollers to close on one another, and upon release of the button the rollers separate from one another, such that the rollers return to an open position.

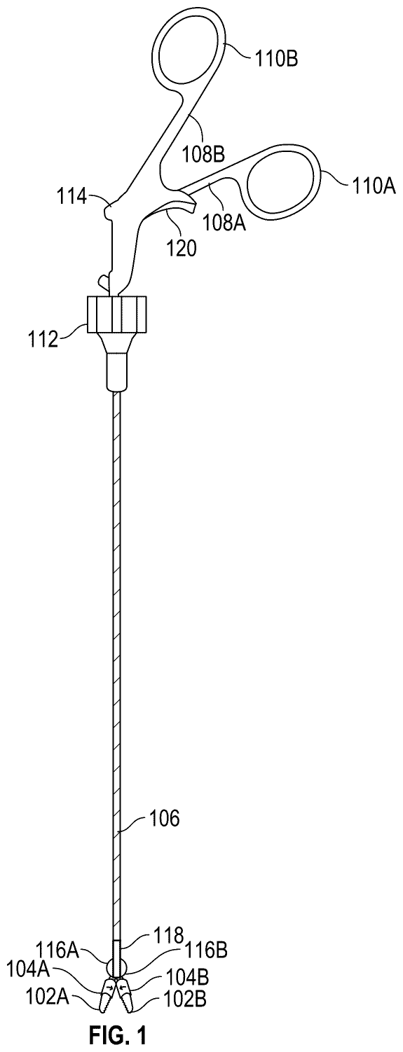

FIG. 1 illustrates a first example apparatus for manipulating suture thread. As illustrated, it can include one or more rollers 104A, 104B, a rotary motor that can be located in the housing 114 and rotates rollers 104A and 104B. In such configurations, the rotary motor can be attached to a shaft 106. Alternatively, in some configurations the rotary motor may not be contained in the housing 114, but instead may be located inside shaft 106. The apparatus further contains jaw members 102A, 102B, a flexible shaft coupler 118, the housing 114 (which can contain the trigger 120) and handles 108A, 108B with rings 110A, 110B for the user's fingers. The rotation knob 112 can adjust the angle of the graspers (i.e., the jaw members 102A, 102B) by rotating the shaft 106 with respect to the housing 114 and/or the handles 108A, 108B. The apparatus preferably functions to manipulate the suture thread in a desired direction. The rollers 104A, 104B individually rotate against a surface in the proximity of surgical site to move suture thread forward. The suture thread is situated between the rollers 104A, 104B and the surface, which may be an opposing roller or a stationary bracing element. That is, in some instances, rather than having the suture thread pulled taut by the rollers 104A, 104B pressing against one another, the suture thread can be pulled taut by a single roller 104A or 104B and an alternative, stationary bracing element. The motion of the roller 104A, 104B and the traction/friction from the opposing surface drives the suture thread forward. The rotary motor provides the rotating driving force that is translated down the shaft 106 to the roller 104A, 104B. The shaft 106 translates the rotational motion from the rotary motor (located in the housing 114) to the roller 104A, 104B. The jaw members 102A, 102B grasp and manipulate the needle, suture thread, or tissue. The trigger 120 toggles the rotary motor to switch it on or off (e.g., when the user pulls the trigger 120 it engages the rotary motor, while when the user releases the trigger 120 it disengages the rotary motor). Since the jaw members 102A, 102B have a hinge to open and close, a flexible shaft coupler 118 allows for the shaft 106 between the rotary motor and the rollers 104A, 104B to bend when the jaw members 102A, 102B are open. When the jaw members 102A, 102B are closed, the shaft 106 is once again straight and can rotate using the rotary motor to move rollers 104A and 104B. The handle 108A, 108B can be used to open and close the jaw members 102A, 102B, and by extension move the rollers 104A, 104B next to one another. During use, the rollers 104A, 104B rotate to pull the suture forward, and the flexible shaft coupler 118 can bend or be straightened as the jaw members 102A, 102B move.

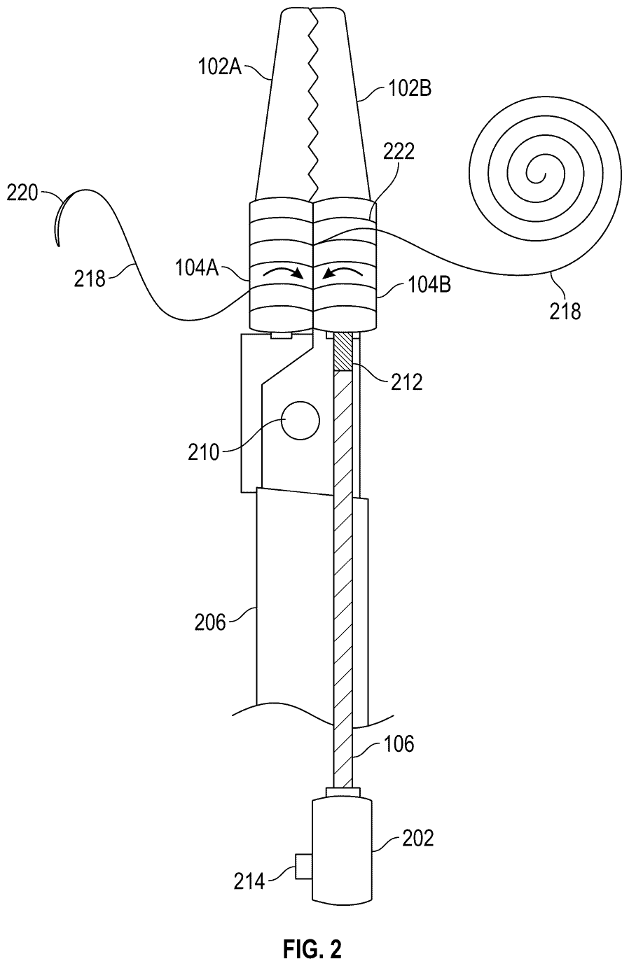

FIG. 2 illustrates an expanded view of the apparatus illustrated in FIG. 1. As illustrated, the rollers 104A, 104B are coupled to a motor embedded in a motor housing 202 using a rotating shaft 106. The rollers 104A, 104B as illustrated are coupled to jaw members 102A, 102B. The rollers' 104A, 104B material can be, for example, rubber, steel, or foam. Alternatively, the rollers' 104A, 104B material may be such that it would provide a desired grip to manipulate suture thread 218 connected to a suturing needle 220. The rollers 104A, 104B may have grooved surface 222 for better traction with the suture thread 218. The rollers 104A, 104B preferably rotate in opposite direction while in flush contact with each other. The suture thread 218 can be placed in between the two rollers 104A, 104B, and the rotation of the rollers 104A, 104B can push (or pull) the suture thread 218 forward. Hinge components 116A and 116B flare out when the grasper is open.

As shown in FIGS. 1 and 2, the apparatus can include a motor embedded in the motor housing 202. The motor preferably functions to rotate the rollers 104A, 104B to manipulate the suture thread 218 in either direction. The suture thread 218 is attached to the suturing needle 220. The motor is configured to cause the rollers 104A, 104B to rotate by spinning a shaft 106. The shaft 106 is enclosed inside a shaft housing 206. The shaft housing 206 is the larger compartment that contains the mechanisms for the laparoscopic suture grasping and for the rotating shaft 106. Since the shaft 106 will be rotating while in use, a protective housing (i.e., the shaft housing 206) is necessary to prevent the shaft 106 from getting caught on nearby tissue. As illustrated, the motor can be configured to engage via a trigger button 214, which controls the engagement of the motor. Once trigger button 214 is depressed, it rotates shaft 106 resulting in 104B and/or 104A to rotate as well.

As shown in FIGS. 1 and 2, the apparatus can include a flexible shaft coupler 212 on the proximal part of the jaw members 102A, 102B. The flexible shaft coupler 212 preferably functions to allow hinge movement of the forceps. The flexible shaft coupler 212 is configured to couple to the shaft 106 leading to the motor housing 202 and the rollers 104A, 104B. The flexible shaft coupler 212 can, for example, be made out of rubber, silicone, or foam. Alternatively, the flexible shaft coupler may be made out of any desired material to allowing the hinge 210 of the jaw members 102A, 102B to move.

As shown in FIGS. 1 and 2, the apparatus can include jaw members 102A, 102B. The jaw members 102A, 102B cooperate with a hinge 210, connecting shaft 106 and a handle (or, as illustrated, motor housing 202 with a trigger button 214) to manipulate objects. The connecting shaft 106 connects the handle (or motor housing 202) to the opposing jaw members 102A, 102B. When the trigger button 214 is depressed, the motor engages, resulting in the rotation of the rollers 104A, 104B. The jaw members 102A, 102B can be made of steel or any desired material to manipulate tissue, suture needle, and suture thread.

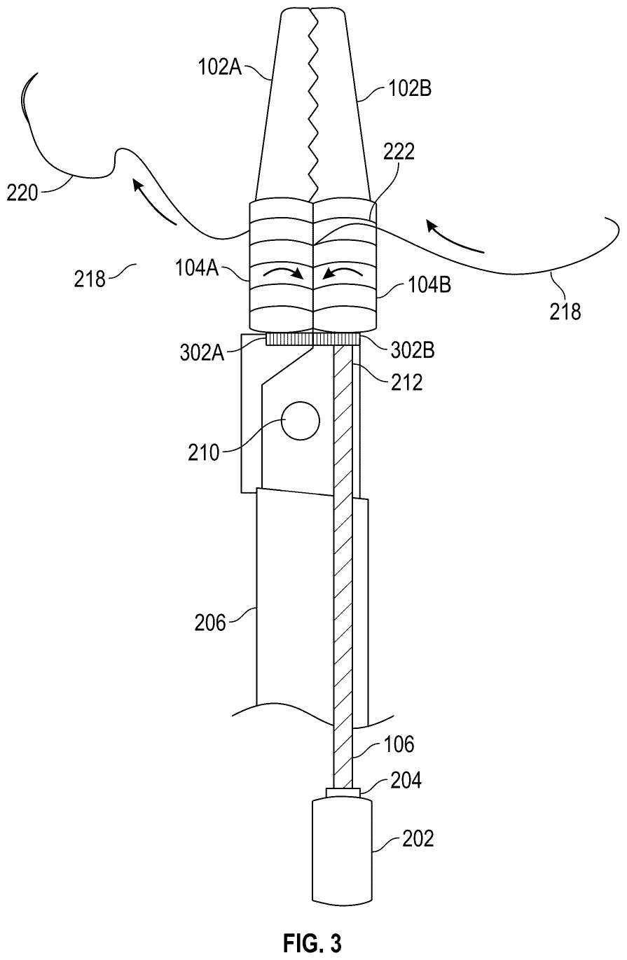

FIG. 3 illustrates a second example apparatus for manipulating suture thread. In this example, the system can mechanically couple the movement of the opposing rollers 104A, 104B. The mechanical coupling can be done using interlocking gears 302A and 302B, where while the motor spins the shaft 106, a first gear 302B spins (along with a first roller 104B), teeth of the first gear 302B couple to teeth a second gear 302A, causing the second gear 302A to spin, while also causing the second roller 104A to spin. The interlocking gears 302A, 302B connect the movement of the roller 104B and the opposing roller 104A so that they move in tandem (in opposite directions). The mechanical coupling of the rollers 104A, 104B moves the suture thread 218 in the desired direction.

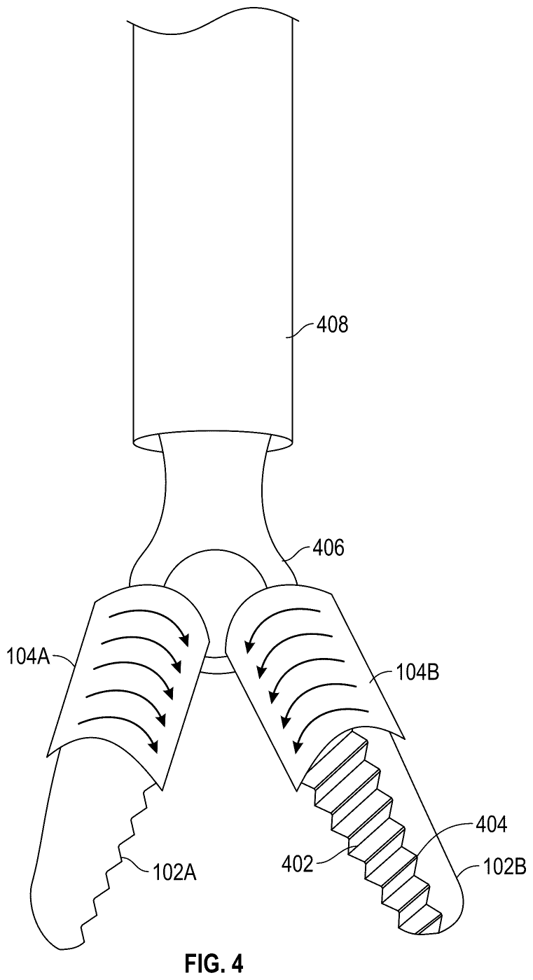

FIG. 4. illustrates a third example of jaw members 102A, 102B and rollers 104A, 104B as used in the third example apparatus, with the jaw members 102A, 102B as illustrated in the open position. As illustrated in FIG. 4, the jaw members 102A, 102B can have teeth 404 which, when the jaw members 102A, 102B are closed, engage/couple with teeth of the other jaw member. In some configurations, such at the illustrated example, the jaw members 102A, 102B can have perpendicular ridges 402 extending across the jaw member in a manner perpendicular to the length of the jaw member. As further illustrated, the jaw members 102A, 102B can be connected via a hinge 406 which connects to a shaft housing 408, the shaft housing 408 containing components used to operate the rollers 104A, 104B and the jaw members 102A, 102B.

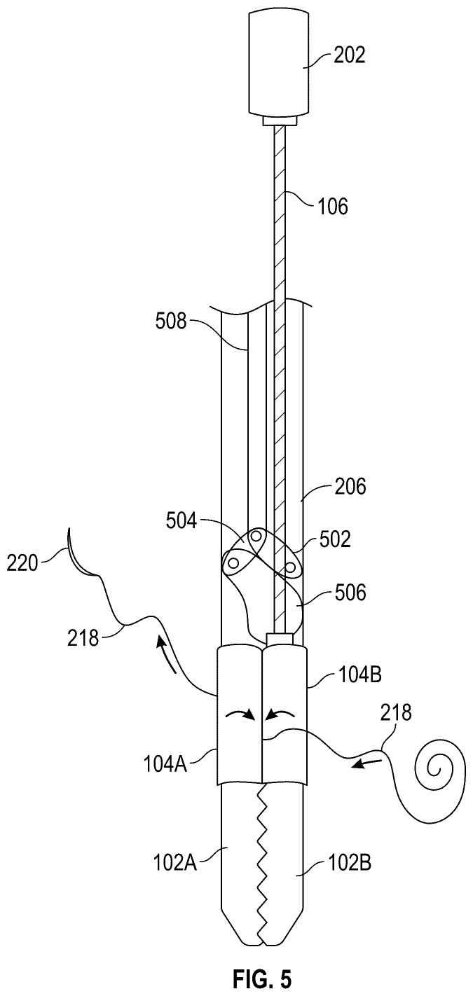

FIG. 5. illustrates a fourth example apparatus for manipulating suture thread 218, and can include rollers 104A, 104B located on the distal portion (i.e., immediately adjacent to the jaw members 102A, 102B) that is immediately above 102 A and 102B without grooves on the surface. The flexible shaft coupler 212 connects the rotating shaft 106 to the roller 104B. Moreover, in this example, within the shaft housing 206 are mechanical components 502, 504, 506, 508. As the user opens the handles, 508 gets pushed down towards the jaw members 102A, 102B, and mechanical components 502, 504 slide apart to push 506 down, which moves (pushes) roller 104B and jaw member 102B to the open position. Upon squeezing and closing the handles, 508 is retracted upward (in this illustration, but otherwise toward the handles), 504 and 502 come together, thereby moving 506 upward, causing roller 104B and jaw member 102B to move to the closed position.

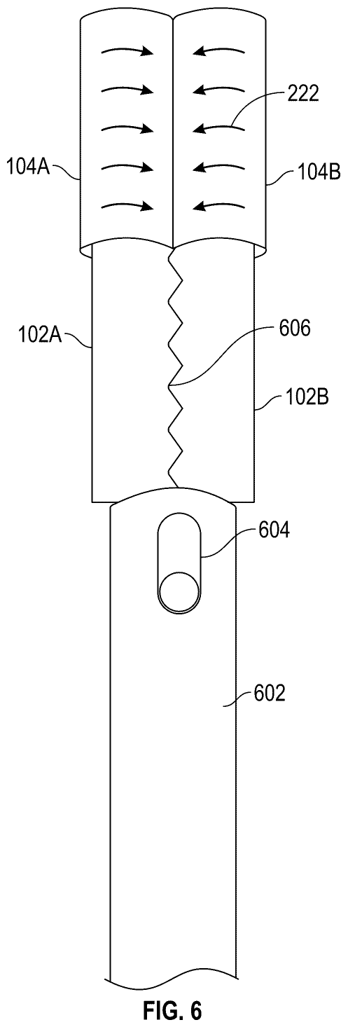

FIG. 6. illustrates a fifth example of jaw members 102A, 102B and rollers 104A, 104B as used in a fourth example apparatus, with rollers 104A, 104B located on the distal portion that is immediately above 102 A and 102B coupled to proximally located jaw members 102A and 102B. As illustrated, the jaw members 102A, 102B are closed, resulting in a tight cohesion 606 between the jaw members 102A, 102B. In this example, a shaft housing 602 contains a switch 604, which can be used to engage the rollers 104A, 104B. The opening and closing of the jaws is operated by the handles 108A and 108B.

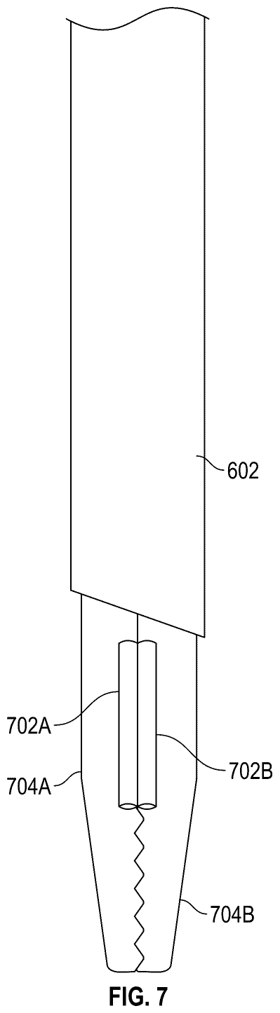

FIG. 7. illustrates a sixth example apparatus for manipulating suture thread, with the apparatus including opposing rollers 702A, 702B encased within the jaw members 704A, 704B. As in previously illustrated configurations, the jaws members 704A, 704B form a tight cohesion 606 when closed. Also, there is a shaft housing 602.

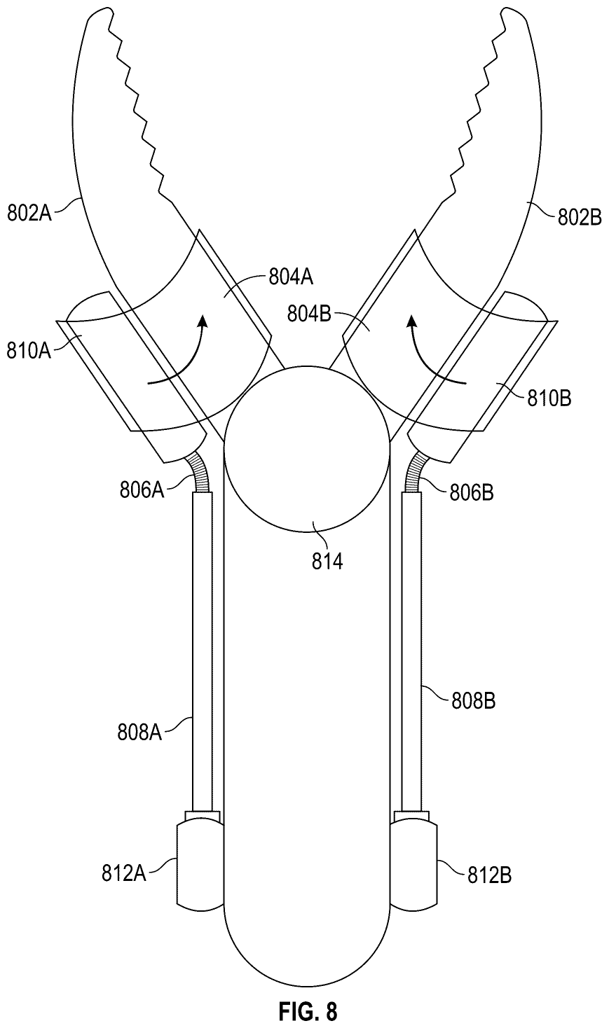

FIG. 8. illustrates a seventh example apparatus for manipulating suture thread, with the apparatus including rollers 810A, 810B coupled to jaw members 802A, 802B, where the rollers 810A, 810B move thin rubber sheets 804A, 804B around the jaw members 802A, 802B. The rollers 810A, 810B are coupled to a flexible shaft coupler 806A, 806B, which in turn is coupled to a shaft 808A, 808B which is coupled to the motor 812A, 812B. Thus, in the illustrated example, there are two motors 812A, 812B, each of which can be independently engaged according to specific needs. In addition, the jaw members 802A, 802B are coupled to a hinge 814, allowing the jaws to open and close as previously described.

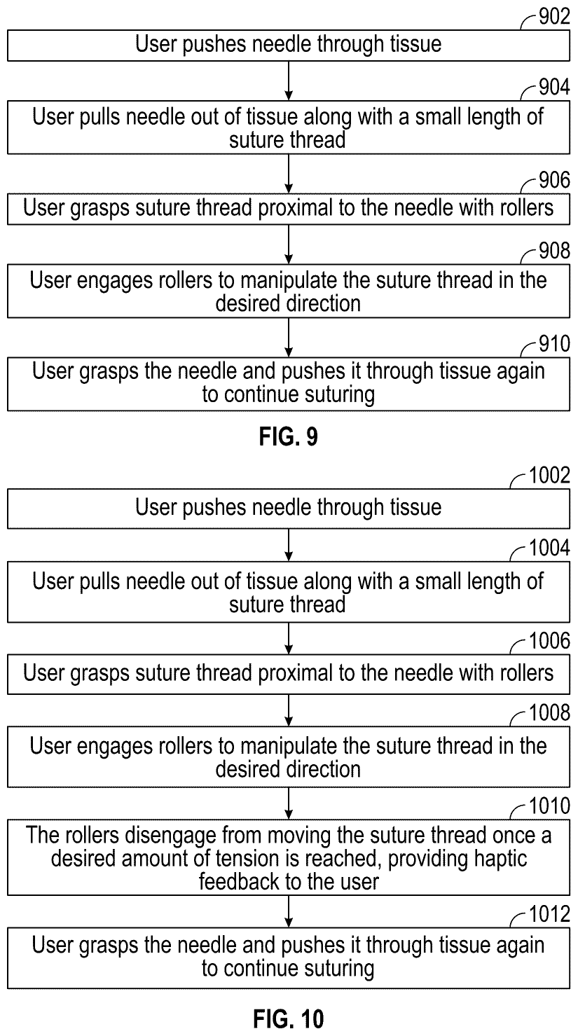

FIG. 9 illustrates a first exemplary method embodiment. As illustrated, the method can include a user pushing the needle through the tissue (902), the user pulling needle out of the tissue along with a length of suture thread (904), and the user grasping the suture thread proximal to the needle with rollers (906). The user can then engage the rollers to manipulate the suture thread in the desired direction (908), and the user grasps the needle to drive it through tissue again to continue suturing (910). The method preferably functions to manipulate the suture thread in the desired direction. The preferred method is preferably used for endoscopic and/or minimally invasive surgery, but can additionally or alternatively be used for any desired applications, clinical or otherwise. The method can be configured and/or adapted to function for a desired application.

FIG. 10 illustrates a second exemplary method embodiment can include pushing the needle through the tissue (1002), pulling needle out of the tissue along with a length of suture thread (1004), grasping the suture thread proximal to the needle with rollers (1006), engaging rollers to manipulate the suture thread in the desired direction (1008), the rollers disengage from moving the suture thread once a desired amount of tension is reached, providing haptic feedback to the user (1010), and grasping the needle to drive it through tissue again to continue suturing in step (1012).

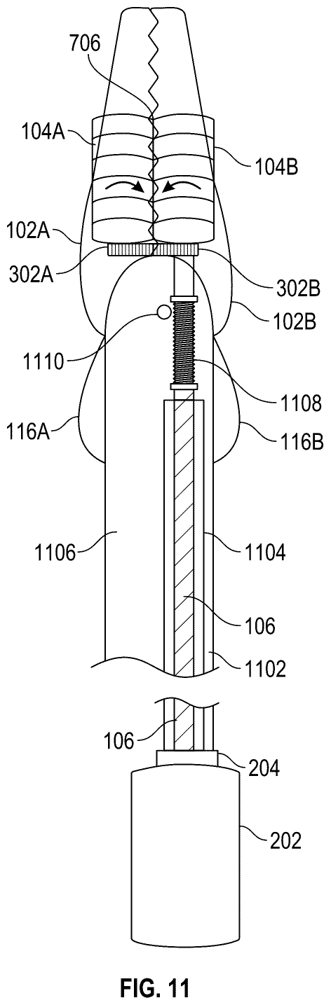

FIG. 11 illustrates an eighth example apparatus for manipulating suture thread. The apparatus can include rollers 104A, 104B that are separate and detachable from the apparatus (e.g., a laparoscopic instrument) and provide a force on the suture thread that may be powered by interlocking gears 302A, 302B or any other mechanism which transfers motion to manipulate the suture thread in the desired direction. A flexible shaft coupler 1108 can be coupled to a shaft 106, which may be encased in a protective sheath 1104. The protective sheath 1104 can be necessary in a design where the device is removable from the jaw members 102A, 102B. Since the shaft 106 is rotating outside of the shaft housing 1106, a protective sheath 1104 can be helpful to prevent the shaft 106 from getting caught on nearby tissue. The motor housing 202 (in which is located the motor) may be located anywhere on or near the apparatus. 1102 indicates a break in the illustration, after which the shaft 106 and protective sheath 1104 may extend for a longer length before connecting to the motor housing 202. The hinge 1110 provides jaw members 102A, 102B a point around which they can pivot.

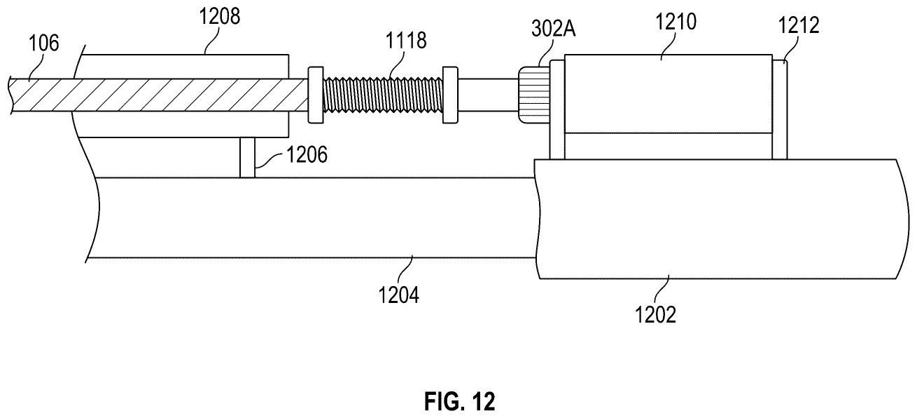

FIG. 12 illustrates a ninth example apparatus for manipulating suture thread. In this example, the apparatus can include one or more rollers 1210, a stabilizer 1212, interlocking gears 302A, flexible shaft coupler 1118, shaft 106, protective sheath 1208, and attaching point 1206, which are separate and detachable from the jaw members 1202 and elongated body 1204. The rollers may be powered by interlocking gears 302A or any other material which transfers motion to manipulate the suture thread in the desired direction. A flexible shaft coupler 1118 is coupled to a shaft 106, which may be encased in a protective sheath 1208.

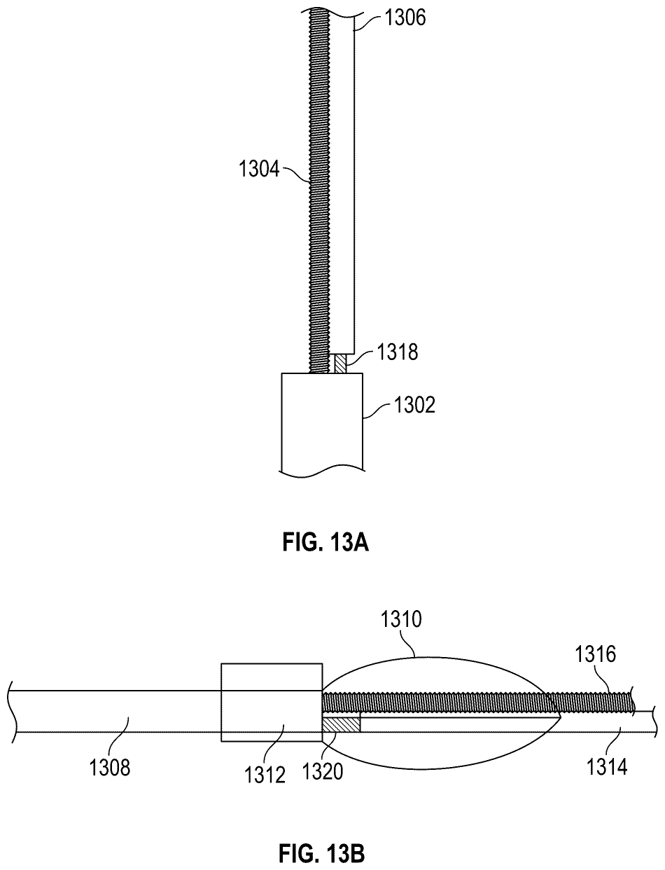

FIGS. 13A and 13B illustrate a tenth example apparatus for manipulating suture thread. The apparatus can include a roller 1304 or 1316, which may be grooved for better traction, bracing element 1306 or 1314, flexible shaft component 1318 or 1320, and a motor 1312 or 1302 that is separate and detachable from the jaw members 1310 or elongated body 1308. The assembly may include a motor embedded in 1302 or 1312, which transfers motion onto a roller 1304 or 1316, and a bracing element 1314 or 1306 to provide friction. A bracing element 1314 or 1306 is a component made of firm material that provides a surface against which the roller would rotate. This surface provides the traction/friction to pull the suture thread forward as the roller rotates. The roller 1304 or 1316 or the bracing element 1306 or 1314 may also have grooves on the surface.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Use of language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure. For example, unless otherwise explicitly indicated, the steps of a process or method may be performed in an order other than the example embodiments discussed above. Likewise, unless otherwise indicated, various components may be omitted, substituted, or arranged in a configuration other than the example embodiments discussed above.

Further aspects of the present disclosure are provided by the subject matter of the following clauses.

An apparatus for manipulating suture thread in surgery, comprising: a rotary motor; a housing with the rotary motor enclosed therein; a pair of rollers; a shaft having a first end and a second end, the shaft coupled at the first end to the pair of rollers and at the second end to the housing; a pair of jaws, each jaw in the pair of jaws respectfully coupled to a roller in the pair of rollers, such that when pressed together each jaw in the pair of jaws engages with the other jaw, and each roller in the pair of rollers is pressed against the other roller; a trigger assembly sitting on the shaft which, when engaged, causes the rotary motor to move the pair of jaws and the pair of rollers towards one another, and a handle assembly coupled to the shaft, wherein when the handle assembly is squeezed and the trigger assembly is pulled: the rotary motor is engaged and rotates the shaft, causing at least one roller in the pair of rollers to manipulate a suture thread in a direction.

The apparatus of any preceding clause, further comprising: a detachable battery compartment, the detachable battery compartment connected to the housing with the rotary motor.

The apparatus of any preceding clause, further comprising: a pair of interlocking gears, wherein a first interlocking gear in the pair of interlocking gears is coupled to the shaft and a first roller in the pair of rollers, and is integrated via first gear teeth with second gear teeth of a second interlocking gear in the pair of interlocking gears, the second interlocking gear coupled to a second roller in the pair of rollers, such that when the when the rotary motor is engaged and rotates the shaft, the first interlocking gear rotates with the shaft, causing the second interlocking gear to rotate in an opposite direction to that of the first interlocking gear, and causing the pair of rollers to rotate

The apparatus of any preceding clause, wherein the pair of rollers rotate in opposite direction, wherein: the suture thread is placed in between the pair of rollers; and the rotation of rollers pushes the suture thread forward.

The apparatus of any preceding clause, further comprising: a hinge; and a handle which, when squeezed, causes the pair of jaws and the pair of rollers to move towards one another, wherein the pair of jaws cooperate with the hinge.

The apparatus of any preceding clause, wherein: the pair of rollers are detachable from the apparatus.

The apparatus of any preceding clause, further comprising: a detachable battery compartment connected to the housing with the rotary motor, wherein the detachable battery compartment is electrically connected to the rotary motor, such that a battery within the detachable battery compartment can power the rotary motor.

The apparatus of any preceding clause, wherein the suture thread is connected to a suture needle which has passed through tissue, such that at least a portion of the suture thread is engaged with tissue.

The apparatus of any preceding clause, wherein the suture thread is situated between a roller in the pair of rollers and a surface, wherein the surface is a stationary bracing element.

The apparatus of any preceding clause, wherein the shaft translates rotational motion from the rotary motor to the pair of rollers.

The apparatus of any preceding clause, wherein the trigger assembly toggles the rotary motor to switch the rotary motor on or off.

An apparatus, comprising: a pair of rollers; a shaft having a first end and a second end, the shaft coupled at the first end to the pair of rollers and at the second end to a handle assembly; a pair of jaws, each jaw in the pair of jaws respectfully coupled to a roller in the pair of rollers, such that when pressed together each jaw in the pair of jaws engages with the other jaw, and each roller in the pair of rollers is pressed against the other roller; a trigger assembly sitting on the shaft which, when engaged, causes the a motor to move the pair of jaws and the pair of rollers towards one another, and the handle assembly coupled to the shaft, wherein when the handle assembly is squeezed and the trigger assembly is pulled: the motor is engaged and rotates the shaft, causing at least one roller in the pair of rollers to manipulate a suture thread in a direction.

The apparatus of any preceding clause, further comprising: a detachable battery compartment, the detachable battery compartment connected to the motor.

The apparatus of any preceding clause, further comprising: a pair of interlocking gears, wherein a first interlocking gear in the pair of interlocking gears is coupled to the shaft and a first roller in the pair of rollers, and is integrated via first gear teeth with second gear teeth of a second interlocking gear in the pair of interlocking gears, the second interlocking gear coupled to a second roller in the pair of rollers, such that when the when the motor is engaged and rotates the shaft, the first interlocking gear rotates with the shaft, causing the second interlocking gear to rotate in an opposite direction to that of the first interlocking gear, and causing the pair of rollers to rotate.

The apparatus of any preceding clause, wherein the pair of rollers rotate in opposite direction, wherein: the suture thread is placed in between the pair of rollers; and the rotation of rollers pushes the suture thread forward.

The apparatus of any preceding clause, further comprising: a hinge; and a handle which, when squeezed, causes the pair of jaws and the pair of rollers to move towards one another, wherein the pair of jaws cooperate with the hinge.

The apparatus of any preceding clause, wherein: the pair of rollers are detachable from the apparatus.

The apparatus of any preceding clause, further comprising: a detachable battery compartment electrically connected to the motor, such that a battery within the detachable battery compartment can power the motor.

The apparatus of any preceding clause, wherein the suture thread is connected to a suture needle which has passed through tissue, such that at least a portion of the suture thread is engaged with tissue.

The apparatus of any preceding clause, wherein the suture thread is situated between a roller in the pair of rollers and a surface, wherein the surface is a stationary bracing element.

We claim:

1. An apparatus for manipulating suture thread in surgery, comprising:
a rotary motor;
a housing with the rotary motor enclosed therein;
a pair of rollers;
a shaft having a first end and a second end, the shaft coupled at the first end to the pair of rollers and at the second end to the housing;
a pair of jaws, each jaw in the pair of jaws respectfully coupled to a roller in the pair of rollers, such that when pressed together each jaw in the pair of jaws engages with the other jaw, and each roller in the pair of rollers is pressed against the other roller;
a trigger assembly sitting on the shaft which, when engaged, causes the rotary motor to move the pair of jaws and the pair of rollers towards one another, and
a handle assembly coupled to the shaft,
wherein when the handle assembly is squeezed and the trigger assembly is pulled:
the rotary motor is engaged and rotates the shaft, causing at least one roller in the pair of rollers to manipulate a suture thread in a direction.

2. The apparatus of claim 1, further comprising:
a detachable battery compartment, the detachable battery compartment connected to the housing with the rotary motor.

3. The apparatus of claim 1, further comprising:
a pair of interlocking gears, wherein a first interlocking gear in the pair of interlocking gears is coupled to the shaft and a first roller in the pair of rollers, and is integrated via first gear teeth with second gear teeth of a second interlocking gear in the pair of interlocking gears,
the second interlocking gear coupled to a second roller in the pair of rollers,
such that when the when the rotary motor is engaged and rotates the shaft, the first interlocking gear rotates with the shaft, causing the second interlocking gear to rotate in an opposite direction to that of the first interlocking gear, and causing the pair of rollers to rotate.

4. The apparatus of claim 1, wherein the pair of rollers rotate in opposite direction, wherein:
the suture thread is placed in between the pair of rollers; and
the rotation of rollers pushes the suture thread forward.

5. The apparatus of claim 1, further comprising:
a hinge; and
a handle which, when squeezed, causes the pair of jaws and the pair of rollers to move towards one another,
wherein the pair of jaws cooperate with the hinge.

6. The apparatus of claim 1, wherein:
the pair of rollers are detachable from the apparatus.

7. The apparatus of claim 1, further comprising:
a detachable battery compartment connected to the housing with the rotary motor, wherein the detachable battery compartment is electrically connected to the rotary motor, such that a battery within the detachable battery compartment can power the rotary motor.

8. The apparatus of claim 1, wherein the suture thread is connected to a suture needle which has passed through tissue, such that at least a portion of the suture thread is engaged with tissue.

9. The apparatus of claim 1, wherein the suture thread is situated between a roller in the pair of rollers and a surface, wherein the surface is a stationary bracing element.

10. The apparatus of claim 1, wherein the shaft translates rotational motion from the rotary motor to the pair of rollers.

11. The apparatus of claim 1, wherein the trigger assembly toggles the rotary motor to switch the rotary motor on or off.

12. An apparatus, comprising:
a pair of rollers;
a shaft having a first end and a second end, the shaft coupled at the first end to the pair of rollers and at the second end to a handle assembly;
a pair of jaws, each jaw in the pair of jaws respectfully coupled to a roller in the pair of rollers, such that when pressed together each jaw in the pair of jaws engages with the other jaw, and each roller in the pair of rollers is pressed against the other roller;
a trigger assembly sitting on the shaft which, when engaged, causes the a motor to move the pair of jaws and the pair of rollers towards one another, and
the handle assembly coupled to the shaft,
wherein when the handle assembly is squeezed and the trigger assembly is pulled:
the motor is engaged and rotates the shaft, causing at least one roller in the pair of rollers to manipulate a suture thread in a direction.

13. The apparatus of claim 12, further comprising:
a detachable battery compartment, the detachable battery compartment connected to the motor.

14. The apparatus of claim 12, further comprising:
a pair of interlocking gears, wherein a first interlocking gear in the pair of interlocking gears is coupled to the shaft and a first roller in the pair of rollers, and is integrated via first gear teeth with second gear teeth of a second interlocking gear in the pair of interlocking gears, the second interlocking gear coupled to a second roller in the pair of rollers,
such that when the when the motor is engaged and rotates the shaft, the first interlocking gear rotates with the shaft, causing the second interlocking gear to rotate in an opposite direction to that of the first interlocking gear, and causing the pair of rollers to rotate.

15. The apparatus of claim 12, wherein the pair of rollers rotate in opposite direction, wherein:
the suture thread is placed in between the pair of rollers; and
the rotation of rollers pushes the suture thread forward.

16. The apparatus of claim 12, further comprising:
a hinge; and
a handle which, when squeezed, causes the pair of jaws and the pair of rollers to move towards one another,
wherein the pair of jaws cooperate with the hinge.

17. The apparatus of claim 12, wherein:
the pair of rollers are detachable from the apparatus.

18. The apparatus of claim 12, further comprising:
a detachable battery compartment electrically connected to the motor, such that a battery within the detachable battery compartment can power the motor.

19. The apparatus of claim 12, wherein the suture thread is connected to a suture needle which has passed through tissue, such that at least a portion of the suture thread is engaged with tissue.

20. The apparatus of claim 12, wherein the suture thread is situated between a roller in the pair of rollers and a surface, wherein the surface is a stationary bracing element.
